(19) United States
(12) Patent Application Publication
(10) Pub. No.: US 2008/0281317 A1
(43) Pub. Date: Nov. 13, 2008

(54) Endometrial Ablation catheter

(76) Inventor: Fred Gobel, Wilhelmsfeld (DE)

Correspondence Address: KIMBERLY-CLARK WORLDWIDE, INC.;Catherine E. Wolf, 401 NORTH LAKE STREET, NEENAH, WI, 54956, US

(21) Appl. No.: 11/801,645

(22) Filed: May 10, 2007

(51) Int. Cl. A61B 18/18

(52) U.S. Cl. 606/41

(57) ABSTRACT

An apparatus for ablating tissue with the uterine cavity is provided. The apparatus includes an ablation catheter and an expansible member. The expansible member is attached to the ablation catheter and it has a residual volume greater than the volume of the uterine cavity and exhibits residual dimensions greater than the anatomical dimensions of the uterine cavity.

### BACKGROUND

Endometrial Ablation is a procedure that removes the lining of the uterus. The procedure is frequently performed for women who have prolonged and uncontrolled menstrual bleeding when the bleeding has not responded to other treatments.

Traditionally, endometrial ablation may be performed utilizing a variety of instruments and techniques. These techniques include endometrial ablation by laser beam (laser thermal ablation); heat (thermal ablation); utilizing radiofrequency or a balloon filled with saline solution that has been heated (thermal balloon ablation); utilization of a resectoscope with a loop or rolling ball electrode (electrical ablation); or freezing.

Although, there are a number of techniques through which thermal ablation may be achieved, many of these prior techniques are either very difficult to master; do not evenly ablate the uterine lining; or pose a risk uterine burns or infection.

Electrical ablation utilizing a resectoscope involves placing a wire loop inside the uterus and transmitting high-frequency electrical energy to cut or coagulate tissue. This method has the advantage of being able to additionally remove polyps and fibroids at the time of ablation and it generally provides excellent results. However, the technique is very difficult to master.

Laser thermal ablation, thermal ablation, and thermal balloon ablation are also techniques for ablation. However, they present serious risks to the patient and are not always effective. In this regard, in prior art laser thermal ablation, thermal ablation, and thermal balloon ablation, heat and/or pressure is applied to the uterus, usually by transmitting the heat through a balloon or balloon-like structure. Because these prior art techniques depend on the balloon to expand to reach all corners of the uterine cavity, there is a risk or the balloon thinning out and developing tears during expansion because of the high filling pressure required to expand the balloon and the correspondingly high pressure exerted on the outer layer of the uterus. This could allow a passageway for pressurized heat or liquid heat to come into direct contact with the uterine lining and cause damage such uterine or intestinal perforation, burns to the uterus or the surface of the bowel, and tearing of the uterus.

Thus, there remains a need for an effective method of thermal ablation which is uncomplicated to perform and which does not pose serious risk of injury to the patient.

### SUMMARY OF THE INVENTION

The present invention provides for an apparatus for ablating tissue in a uterine cavity. The apparatus includes a catheter having a distal end, a proximal end, a catheter body, a catheter tip, one or more openings on the catheter body, and one of more fixation points. The apparatus also includes an expansible member having an outer surface, an interior surface, a distal end, and a proximal end, wherein upon introduction of inflation media into the expansible member, the expansible member defines a volume; wherein the expansible member is adapted to attach to the catheter at the one or more fixation points so that under inflation, the distal end of the expansible member encloses the catheter tip; and wherein the expansible member has a residual volume greater than the volume of the uterine cavity and exhibits residual dimensions greater than the anatomical dimensions of the uterine cavity. In this regard, the residual volume and the residual dimensions of the expansible member may be between about 20% and 60% greater than the volume and anatomical dimensions of the uterine cavity, respectively. The one or more fixation points may include one of more distal fixation points and one or more proximal fixation points. Further, the catheter may include one or more lumens.

Desirably, the expansible member is inflated through the one or more openings of the catheter. This inflation is accomplished by the introduction of gas, heated liquid, or a combination of both through the one or more openings in the catheter body.

Desirably, upon inflation, the outer surface of the expansible member generates substantially even pressure against all interior portions of the uterine cavity including corneal areas without stretching or entering a state of distension. In this regard, in response to inflation, it is desirable that the expansible member expand zero percent.

For example, the expansible member, in response to inflation, should exhibit behavior similar to a hot air balloon or parachute. In this regard, little or no stretching or distension of the expansible member is required to effect inflation.

Conversely, a toy balloon of the type which is typically inflated with the mouth, will stretch or distend in order to inflate.

The apparatus may include a hysteroscope partially enclosing the catheter or adjacent to the catheter. The apparatus may also incorporate a puls-oximeter sensor on the outer surface of the expansible member.

The expansible member may have a shape corresponding to the shape of the uterine cavity or may be conically, cylindrically, or spherically shaped and may be a balloon. Additionally, upon inflation a pressure ranging from about 120 mmHg to about 200 mmHg is exerted upon the interior surface of the expansible member.

Desirably, the expansible member may include a polyurethane material and may have a thickness ranging from about 5 microns to about 30 microns. Additionally, the expansible member may be pre-shaped prior to attachment to the catheter.

Another aspect of the invention addresses a method of ablating tissue in the uterine cavity. The method includes the steps of: a) inserting a catheter into the uterine cavity, the catheter having a distal end; a proximal end; a catheter body; a catheter tip; one or more openings on the catheter body; and one of more fixation points, the catheter further having an expansible member connected thereto at the one or more fixation points, the expansible member having an outer surface; an interior surface; a distal end; and a proximal end, the expansible member having a residual volume greater than the volume of the uterine cavity and exhibiting residual dimensions greater than the anatomical dimensions of the uterine cavity. In this regard, the residual volume and the residual dimensions of the expansible member may be between about 20% and 60% greater than the volume and anatomical dimensions of the uterine cavity, respectively; b) introducing a fluid into the proximal end of the catheter so that the fluid travels through the one or more openings into the expansible member so that the expansible member defines a volume which results in inflation of the expansible member without distension of the expansible member, wherein the outer surface of the expansible member exerts sufficient pressure against the uterine tissue to prevent blood from flowing into the uterine tissue; and c) deflating the expansible member and removing the catheter from the uterine cavity between about 5 and about 20 minutes after completion of introduction of fluid into the proximal end of the catheter.

A further aspect of the invention addresses a method for providing a system for ablating tissue in a uterine cavity. The method includes the step of providing an apparatus having a catheter having a distal end, a proximal end, a catheter body, a catheter tip, one or more openings on the catheter body, and one of more fixation points; and an expansible member having an outer surface, an interior surface, a distal end, and a proximal end, wherein upon introduction of inflation media into the expansible member, the expansible member defines a volume; wherein the expansible member is adapted to attach to the catheter at the one or more fixation points so that under inflation, the distal end of the expansible member encloses the catheter tip; and wherein the expansible member and wherein the expansible member has a residual volume greater than the volume of the uterine cavity and exhibits residual dimensions greater than the anatomical dimensions of the uterine cavity. In this regard, the residual volume and the residual dimensions of the expansible member may be between about 20% and 60% greater than the volume and anatomical dimensions of the uterine cavity, respectively. The method further includes the step of providing instructions for inserting the catheter into the uterine cavity. The method further includes the step of providing means for introduction of fluid into the proximal end of the catheter so that the fluid travels through the one or more openings into the expansible member so that the expansible member defines a volume which results in inflation of the expansible member without distension of the expansible member, wherein the outer surface of the expansible member exerts sufficient pressure against the uterine tissue to prevent blood from flowing into the uterine tissue. The method further includes yet another step, which is providing means for deflating the expansible member and removing the catheter from the uterine cavity between about 5 and about 20 minutes after completion of introduction of fluid into the proximal end of the catheter.

### BRIEF DESCRIPTION OF DRAWINGS

FIG. 1 is a view of an ablation catheter having a conically shaped expansible member.

FIG. 2 is a view of an ablation catheter having a cylindrically shaped expansible member.

FIG. 3 is a view of an ablation catheter inside a uterine cavity. The ablation catheter in partially enclosed by a hysteroscope and the expansible member includes a puls-oximeter sensor on its outer surface.

FIG. 4 is a view of an ablation catheter inside the uterine cavity.

FIG. 4A is an enlarged view of random folds within the expansible member.

FIG. 5 is a view of an ablation catheter having an expansible member connected to the catheter at one fixation point.

### DEFINITIONS

“Residuality” or “residual dimensions” refers to the percentage by which the dimensional properties or dimensionally based properties (length, width, or volume) of an expansible member is greater than the dimensional properties of the cavity in which it is adapted to be inserted and inflated. For example, an expansible member may have a volume that may be desirably from about 5 to 100 percent, more desirably 20 to 60 percent greater than the volume of the cavity in which it is adapted to be inserted and inflated.

### DETAILED DESCRIPTION

The ablation catheter of the present invention provides for advanced ablation catheters and techniques of thermal ablation which are based on a residually dimensioned microthin expansible member material. This material enables the expansible member to reach all areas of the uterus, including the corneal areas, without unnecessarily high filling pressure and with substantially homogenous force exertion upon the lining of the uterine cavity. The ablation catheter is suitable thermal ablation, pressure ablation without a thermal component, or combined thermal plus pressure ablation.

The invention will be described with reference to the following description and figures which illustrate certain embodiments. It will be apparent to those skilled in the art that these embodiments do not represent the full scope of the invention which is broadly applicable in the form of variations and equivalents as may be embraced by the claims appended hereto. Furthermore, features described or illustrated as part of one embodiment may be used with another embodiment to yield still a further embodiment. It is intended that the scope of the claims extend to all such variations and embodiments.

In the interests of brevity and conciseness, any ranges of values set forth in this specification contemplate all values within the range and are to be construed as support for claims reciting any sub-ranges having endpoints which are whole number values within the specified range in question. By way of a hypothetical illustrative example, a disclosure in this specification of a range of from 1 to 5 shall be considered to support claims to any of the following ranges: 1-5; 1-4; 1-3; 1-2; 2-5; 2-4; 2-3; 3-5; 3-4; and 4-5.

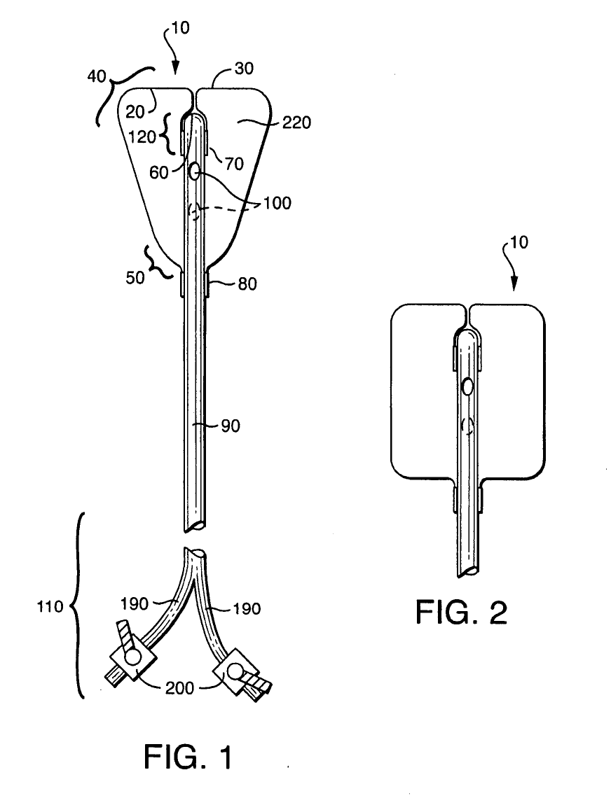

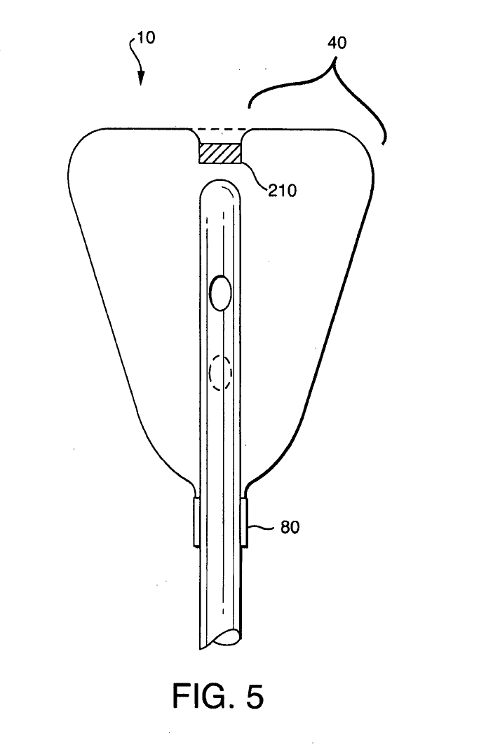

Referring to FIGS. 1, 2, 4 and 5, an apparatus for ablating tissue in a uterine cavity is provided. The apparatus includes a catheter having a catheter body 90, a distal end 40, a proximal end 110, a tip 60, one or more openings 100, and one or more distal 70 and/or proximal 80 fixation points. The catheter may include one or lumens inside the catheter body 90 which may be connected to the one or more openings 100. Additionally, the catheter may incorporate one or more fluid supply lines 190 at its proximal end 110, the fluid supply lines having one or more stopcocks 200 for regulating the ingress and egress of liquid into the catheter body and/or the one or more lumens of the catheter body 90.

The catheter may comprise any flexible material which is compatible with the uterine cavity and does not cause irritation of the cavity. These materials include, but are not limited to, latex, silicone, polyvinyl chloride, polyurethane or polytetrafluoroethylene.

The catheter may be manufactured by any method known in the art of manufacturing catheter tubing. It is desirable, however, to manufacture relatively thin catheter tubing so that the lumen for ingress and egress of fluids may be enlarged. A non-limiting example of a catheter manufacturing process suitable for this purpose is the coextrusion of two tubes (coextrusion being a process known and understood by those having skill in the manufacture of extrude tubing), an outer tube and an inner tube, the inner tube being thinner and harder than the outer tube. Additional, non-limiting examples include utilizing reinforcement inserts such as wire inside the shaft of the catheter, utilizing a spiral wound reinforcement, or utilizing a stabilizing mesh incorporated into the wall of the catheter shaft.

Referring again to FIGS. 1, 2, 4 and 5, the apparatus also include an expansible member 10 attached to the catheter at one or more fixation points. The expansible member includes an outer surface 30, an interior surface 20, a distal end 40, and proximal end 50. The expansible member may be attached to the one or more fixation points utilizing gluing, welding, or any other known method in the art for attaching an expansible member to a catheter. Desirably, the expansible member is a balloon. The expansible may be desirably pre-formed or may be alternatively formed by methods such as blow-molding, dip coating, or spin coating.

When the expansible member is pre-formed it is preformed to exhibit a residual volume between about 5 to about 100 percent, more desirably between about 20 to about 60 percent greater than the residual volume of the uterine cavity. In this regard, it is preformed to exhibit dimensions from between about 5 to about 100 percent, more desirably 20 percent to about 60 percent beyond the anatomical dimensions of the uterine cavity. While these anatomical dimensions are variable, they may be readily determined by one of ordinary skill in the art utilizing conventional techniques.

The preformed expansible member is then attached to the catheter shaft at one or more fixation points 70, 80. When there are two or more fixation points, at least one fixation point 70 is located in a location on the catheter that is in closer proximity to the distal end 120 of the catheter than the one or more openings, and at least one fixation point 80 is located in a location on the catheter that is in closer proximity to the proximal end 110 of the catheter than the one or more openings. Alternatively, when there is only one fixation point 80, the open ends of the expansible member near the distal end of the expansible member may be glued or welded together 210 and desirably reverse-folded to the inside of the expansible member.

As an alterative to pre-folding, the expansible member may be constructed by any of the other various techniques well-known to those skilled in the art. For example without limitation, the polymer can be dip-coated on a mandrel that has a defined size and shape, desirably from between about between about 20% to about 60% beyond the anatomical dimensions of the uterine cavity. When removed from the mandrel, the expansible member in its uninflated or collapsed state will assume two dimensions (length and width) of the mandrel without incurring any tensional force in the polymer. This condition, the shape and dimensions of an expansible member in its uninflated, collapsed state after formation by whatever means selected will, for the purpose of this discussion, be called the expansible member's equilibrium dimensions.

The expansible member may also be formed by spin-coating in a hollow mold. When the mold is removed, as in the case of a dip-coated mandrel, the expansible member will assume equilibrium dimensions that are the same as the interior dimensions of the hollow mold.

In addition, expansible members may be formed by injection or blow molding. In this process, a pre-formed length of tubing made of the polymer is placed in a hollow mold having internal dimensions that reflect the desired equilibrium dimensions of the expansible member to be formed. One end of the tube is sealed off and a working fluid is injected into the open end of the tube with sufficient force to cause the working fluid to expand the tubing until the wall of the tubing is in intimate contact with the inner surface of the mold. The polymer is then annealed, if desired, and cooled after which the mold is removed leaving a portion of the tubing as a expansible member that will assume equilibrium dimensions.

The above are but a few methods of forming the expansible member. Others will be apparent to those skilled in the art. All such methods are within the scope of this invention.

Various materials may used to form the expansible member. These materials include, but are not limited to, polyurethane (PU), low-density polyethylene (LDPE), polyvinyl chloride (PVC), polyamid (PA) or polyethylene teraphthalate (PETP). Additionally, copolymer admixtures for modifying the characteristics of the material may be used, for example a low density polyethylene and ethylene-vinylacetate copolymer (LDPE-EVA), or blends of the above mentioned materials (e.g. PU with PVC or PU with PA) would be considered suitable for forming the expansible member. Other materials would also be suitable so long as they exhibit properties enabling them to be processed into expansible members having microthin walls on the order of about 5 to about 30 micrometers, more desirably into a range of between about 5 to about 15 micrometers. Suitable materials should possess properties enabling them to be processed into anchor mechanisms having microthin walls which do not deform elastically to such a degree that they are enabled to slip through the uterine opening into the uterine cavity.

Regardless of the method utilized to form the expansible member or the material used, upon inflation, the expansible member will demonstrate a residual volume between about 5 and about 100 percent, more desirably between about 20 and about 60 percent greater than the volume of the uterine cavity and will exhibit residual dimensions between about 5 and about 100 percent, more desirably between about 20 and about 60 percent greater than the anatomical dimension of the uterine cavity.

Various fluids may be used to inflate the expansible member. The fluid may be a gas or may be liquid, desirably a heated liquid. Desirable gases include, but are not limited to, oxygen. Desirably liquids include, but are not limited to saline.

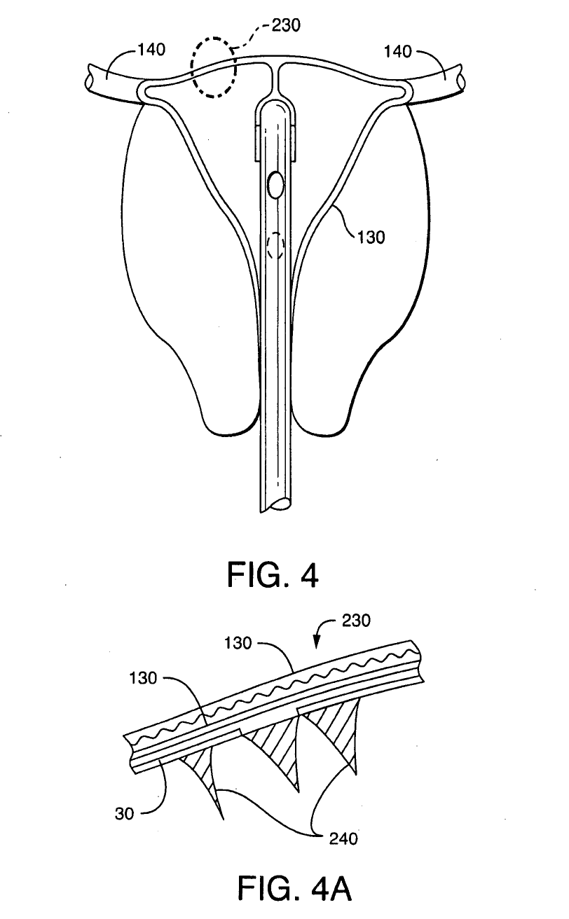

Once the expansible member has been attached to the catheter at the one or more fixation point, it may be inserted into the uterus. Because the expansible member demonstrates residuality (i.e. it exhibits dimensions greater than the dimension of the cavity in which it is inserted) it will have random folds in its structure. These folds 240 are illustrated in FIG. 4a (not drawn to scale) which is an enlarged view 230 of an expansible member having random folds, the expansible member being in communication with uterine cavity tissue 130.

Upon inflation of the expansible member through the one or more openings in the catheter, the folds will partially, but not completely dissipate, allowing the expansible member to desirably reach all corners of the uterine cavity including the corneal areas 140. In this regard, the expansible member may be inflated with a volume 220 of fluid that is between about 40 and about 80 percent of the volume required to distend the expansible member. For purposes of the present disclosure, the terms “distend” and “distension” refer to a degree of inflation of the expansible member beyond its fully inflated state such that the material(s) forming the expansible member stretch or otherwise deform. Importantly, the expansible member, upon inflation, should not reach a state of distension and should retain its original equilibrium dimensions.

The random folds of the expansible member may run in any direction, and thus, for example, may be at transverse or right angles to the catheter shaft axis. Yet, since the foil thickness or expansible member wall is exceptionally thin, from about 5 to about 30 micrometers, upon insertion of the ablation catheter into the uterine cavity and upon removal once fluid is drained through the lumen of the catheter, the expansible member will desirably be in close proximity to the surface of the catheter shaft. In some instances, it may even form hanging sack-like folds when the catheter is inserted or removed. Advantageously, because the material of the expansible member is thin and it desirably is in close proximity to the surface of the catheter shaft upon insertion and removal, easy insertion and removal of the ablation catheter from the uterine cavity may be accomplished without causing tissue trauma to the patient.

Alternatively, the fold formations may be aligned longitudinally with the catheter shaft. In any event, the fold formations permit inflation of the expansible member without distention of the expansible member.

Upon inflation, the expansible member will be inflated to a pressure that is less than the pressure required to distend or stretch the material of the expansible member. This amount of pressure inside the expansible member may be as low as intrauterine pressure but desirably ranges from about 120 mmHg to about 200 mmHg, more desirably from about 150 mmHg to about 180 mmHg. During normal use, an equal amount of pressure should be exerted upon the tissue 130 of the uterine cavity. This amount of pressure will allow the expansible member to contact all portions of the uterine cavity without stretching of the material composing the expansible member. In this regard, the expansible member should not stretch when inflated. While it is contemplated that stretch or elongation of the material of the expansible member might occur, upon inflation, any stretch or elongation should me minimal and at least less than 5 percent, more desirably less than 1 percent, and even more desirably zero percent during normal use. Thus, the expansible member retains its original equilibrium dimensions and does not enter a state of distension. This allows a substantially even amount of pressure to be applied to all portions of the wall of the uterine cavity without forces being unevenly concentrated in one area. According to the invention, the pressure exerted upon the uterine cavity should stop blood from flowing to the tissue of the uterine cavity resulting in necrosis of the tissue after a period of time, desirably between about 5 and 20 minutes.

Conversely, in prior art ablation catheters, the expansible member will expand beyond its normal dimensions and will concentrate forces unevenly upon the uterine cavity. This can result in less effective ablation procedures because portions of the lining of the uterus may be undertreated resulting in less than complete ablation; portions may be overtreated resulting in heat or pressure damage to portions of the lining of the uterus; and some portions may not be treated at all.

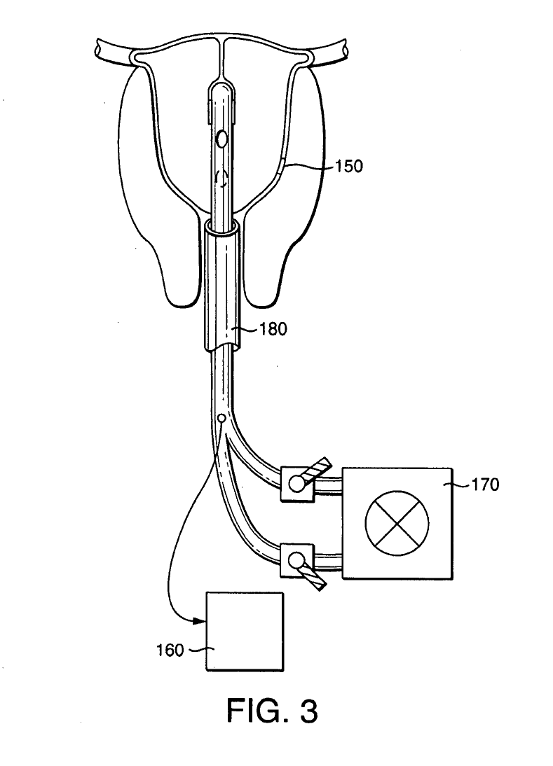

It should be understood that inflation of the expansible member may be accomplished by inflating the expansible member with a gas at between about 120 mmHg to about 200 mmHg, with a heated liquid which may range in temperature between about 50 Celsius and about 80 Celsius, or a combination of heated liquid and gas. In instances, where a combination of heated liquid and gas are utilized to inflate the expansible member, the heated liquid may be transmitted into the expansible member at a pressure of from about 50 mmHg to about 120 mmHg. This may be accomplished by the use of a perfusion device 170 such as the device illustrated in FIG. 3.

The perfusion device 170 may be connected to the proximal end 110 of the catheter shaft 90 and may be used to pump pressurized or unpressurized fluid into the catheter shaft, the fluid adapted to be transmitted through the openings of the catheter shaft into the interior of the expansible member to define a volume 220 within the expansible member. The perfusion device may also be utilized for removing liquid and pressurized gas from the expansible member through the catheter shaft. Alternatively, liquid and/or pressurized gas may be removed from the ablation catheter by opening of the stopcocks 200 present on the proximal end of the ablation catheter.

Additionally, as previously explained, the expansible member will remain inflated inside the uterine cavity until the tissue of the uterine cavity experiences necrosis. This tissue necrosis results from the cessation of blood flow into the tissues the uterine cavity. This cessation may be measured visually utilizing a hysteroscope 180 or may be measured with the use of a puls-oximeter sensor 150 as illustrated in FIG. 3.

In this regard, a hysteroscope may at least partially enclose the catheter or may be adjacent to the catheter upon insertion into the uterine cavity. The hysteroscope allows for viewing of the uterine cavity during the ablation procedure. Thus, after insertion of the ablation catheter into the uterine cavity and inflation of the expansible member within the uterine cavity, the physician performing the procedure will be able to visualize the cessation of blood flow into the tissues of the uterine cavity. This cessation will be indicated by the change of the tissues of the uterine cavity from a pink color to a pale color. Based upon this visual inspection, the physician may be able to calculate or determine when to conclude the ablation procedure.

Additionally, a puls-oximeter device 160 may be used in conjunction with the ablation catheter. In this regard, the puls-oximeter device 160 may be attached to the catheter shaft and may receive signals transmitted from a puls-oximeter sensor attached to the outer surface 30 of the expansible member. The puls-oximeter sensor measures the cessation of blood flow into the tissue of the uterine cavity 130 and transmits a signal conveying this information to the puls-oximeter device where it may be read by the physician. The physician then utilizes this information to calculate or determine when to conclude the ablation procedure.

In addition to the apparatus and method described the present invention encompasses a method of providing a system for ablating tissue in a uterine cavity.

Generally speaking, the system provides for an apparatus, instructions for utilizing the apparatus, and means for utilizing the apparatus. In this regard, the apparatus includes a catheter and an expansible member. The catheter includes a distal end, proximal end, catheter body, catheter tip, one or more openings on the catheter body, and one or more fixation points. The expansible member includes an outer surface, an interior surface, a distal end, and a proximal end.

The instructions provided with the apparatus may include directions for inserting the apparatus inside the uterine cavity. In this regard, the apparatus should be inserted far enough into the uterine cavity such that the distal end of the expansible member is in communication with the tissues of the uterine cavity and is substantially aligned with the corneal regions wherein upon inflation, the expansible member will reach all areas of the uterine cavity including the corneal regions.

The instructions may also include directions for utilizing the apparatus. In this regard, instructions may be provided for inflating the expansible member by introducing a fluid into the proximal end of the catheter utilizing a device such as a perfusion device. These instructions may include a listing of desirable inflation pressures to be maintained within the interior of the expansible member after inflation. Additionally, instructions for deflating the expansible member after a sufficient time, such as, for example 5 to 20 minutes, may be provided.

Further, instructions for use of a hysteroscope for visualizing the cessation of blood flow into the tissues of the uterine cavity may be provided; and instructions for use of a puls-oximeter for measuring the cessation of blood flow into the tissues of the uterine cavity may be provided.

What is claimed is:

1. An apparatus for ablating tissue in a uterine cavity, the apparatus comprising:
a catheter comprising a distal end, a proximal end, a catheter body, a catheter tip, one or more openings on the catheter body, and one of more fixation points; and
an expansible member comprising an outer surface, an interior surface, a distal end, and a proximal end, wherein upon introduction of inflation media into the expansible member, the expansible member defines a volume; wherein the expansible member is adapted to attach to the catheter at the one or more fixation points so that under inflation, the distal end of the expansible member encloses the catheter tip; and wherein the expansible member has a residual volume greater than the volume of the uterine cavity and exhibits residual dimensions greater than the anatomical dimensions of the uterine cavity.

2. The apparatus of claim 1 wherein the expansible member is inflated through the one or more openings of the catheter.

3. The apparatus of claim 2 wherein the inflation of the expansible member is accomplished by the introduction of gas, heated liquid, or a combination of both through the one or more openings.

4. The apparatus of claim 1, wherein the expansible member, upon inflation, does not enter a state of distension.

5. The apparatus of claim 1, wherein, upon inflation, the outer surface of the expansible member generates substantially even pressure against all interior portions of the uterine cavity including corneal areas.

6. The apparatus of claim 1, wherein the catheter is at least partially enclosed by a hysteroscope or is adjacent to a hysteroscope.

7. The apparatus of claim 1, wherein the expansible member further comprises a puls-oximeter sensor on its outer surface.

8. The apparatus of claim 1, wherein the catheter comprises one or more lumens.

9. The apparatus of claim 1, wherein upon inflation, pressure ranging from about 120 mmHg to about 200 mmHg is exerted upon the interior surface of the expansible member.

10. The apparatus of claim 9, wherein upon the inflation, pressure ranging from about 150 mmHg to about 180 mmHg is exerted upon the interior surface of the expansible member.

11. The apparatus of claim 1, wherein the expansible member is conically, cylindrically, or spherically shaped.

12. The apparatus of claim 1, wherein the expansible member has a shape corresponding to the shape of the uterine cavity.

13. The apparatus of claim 1, wherein the one or more fixation points comprise one of more distal fixation points and one or more proximal fixation points.

14. The apparatus of claim 1, wherein the expansible member comprises polyurethane material.

15. The apparatus of claim 1, wherein the thickness of the expansible member ranges from about 5 microns to about 30 microns.

16. The apparatus of claim 1, wherein the expansible member is a balloon.

17. The apparatus of claim 1, wherein the expansible member is pre-shaped prior to attachment to the catheter.

18. The apparatus of claim 1, wherein the expansible member, upon inflation, expands zero percent.

19. The apparatus of claim 1, wherein the expansible member has a residual volume between about 20% and about 60% greater than the volume of the uterine cavity and exhibits residual dimensions between about 20% and about 60% greater than the anatomical dimensions of the uterine cavity.

20. A method for ablating tissue in a uterine cavity, the method comprising:
Inserting a catheter into the uterine cavity, the catheter comprising a distal end; a proximal end; a catheter body; a catheter tip; one or more openings on the catheter body; and one of more fixation points, the catheter further comprising an expansible member connected thereto at the one or more fixation points, the expansible member comprising an outer surface; an interior surface; a distal end; and a proximal end, the expansible member having a residual volume greater than the volume of the uterine cavity and exhibiting residual dimensions greater than the anatomical dimensions of the uterine cavity;
Introducing a fluid into the proximal end of the catheter so that the fluid travels through the one or more openings into the expansible member so that the expansible member defines a volume which results in inflation of the expansible member without distension of the expansible member, wherein the outer surface of the expansible member exerts sufficient pressure against the uterine tissue to prevent blood from flowing into the uterine tissue; and
Deflating the expansible member and removing the catheter from the uterine cavity between about 5 and about 20 minutes after completion of introduction of fluid into the proximal end of the catheter.

21. The method of claim 20, wherein a hysteroscope, which partially encloses or is adjacent to a the catheter, is used to visualize the cessation of blood flow into the tissues of the uterine cavity.

22. The method of claim 20, wherein a puls-oximeter sensor is present on the outer surface of the expansible member and is used to measure the cessation of blood flow into the tissues of the uterine cavity

23. The method of claim 20, wherein upon inflation, pressure ranging from about 120 mmHg to about 200 mmHg is exerted upon the interior surface of the expansible member.

24. The method of claim 20, wherein the fluid is a gas.

25. The method of claim 20, wherein the fluid is a heated liquid.

26. A method for providing a system for ablating tissue in a uterine cavity, the method comprising:
Providing an apparatus comprising
a catheter comprising a distal end, a proximal end, a catheter body, a catheter tip, one or more openings on the catheter body, and one of more fixation points; and
an expansible member comprising an outer surface, an interior surface, a distal end, and a proximal end, wherein upon introduction of inflation media into the expansible member, the expansible member defines a volume; wherein the expansible member is adapted to attach to the catheter at the one or more fixation points so that under inflation, the distal end of the expansible member encloses the catheter tip; and wherein the expansible member has a residual volume greater than the volume of the uterine cavity and exhibits residual dimensions greater than the anatomical dimensions of the uterine cavity;
Providing instructions for inserting the catheter into the uterine cavity;
Providing means for introduction of fluid into the proximal end of the catheter so that the fluid travels through the one or more openings into the expansible member so that the expansible member defines a volume which results in inflation of the expansible member without distension of the expansible member, wherein the outer surface of the expansible member exerts sufficient pressure against the uterine tissue to prevent blood from flowing into the uterine tissue; and
Providing means for deflating the expansible member and removing the catheter from the uterine cavity between about 5 and about 20 minutes after completion of introduction of fluid into the proximal end of the catheter.

27. The method of claim 26, wherein a hysteroscope is provided for visualizing cessation of blood flow into the tissues of the uterine cavity.

28. The method of claim 26, wherein a puls-oximeter sensor is provided for measuring the cessation of blood flow into the tissues of the uterine cavity.
